(19) United States
(12) Patent Application Publication
(10) Pub. No.: US 2021/0251777 A1
(43) Pub. Date: Aug. 19, 2021

(54) Breast Form Adapted for Protruding Abdomen

(71) Applicant: American Breast Care, LP, Marietta, GA (US)

(72) Inventor: Leslie S. Amick, Marietta, GA (US)

(21) Appl. No.: 16/789,560

(22) Filed: Feb. 13, 2020

(51) Int. Cl. A61F 2/52; A61F 2/50

(52) U.S. Cl. CPC A61F 2/52; A61F 2002/501

(57) ABSTRACT

A breast prosthesis includes a back surface having a footprint and a breast-shaped portion extending outwardly from the back surface so that the breast-shaped portion has a shelf. The footprint has a top neckline terminating in a medial point and a bottom shelf edge extending away from the medial point in which the bottom shelf edge of the footprint of the breast prosthesis is a substantially straight line and slopes downwardly away from the medial point when in normal use.

### BACKGROUND OF THE INVENTION

### 1. Field of the Invention

The present invention relates to breast prostheses and, more specifically, to a breast form prosthesis adapted for a user with a protruding belly.

### 2. Description of the Related Art

External silicone breast prostheses are often worn by women to provide a natural look after having had a mastectomy. A typical breast prosthesis 10, as shown in FIG. 1, can include a bag made of a film, such as urethane, into which a silicone compound has been injected. In many such prostheses, the prosthesis is placed in a mold that is complementary in shape to a natural breast and then cured sufficiently for the prosthesis to maintain its shape.

Some prostheses can include two layers of silicone gel encased in three layers of film sealed around the periphery of the form. The layer closest to the wearer can be soft to conform to the wearer's chest while the front layer must be of firmer silicone to support the prosthesis and prevent prosthesis collapse in a bra. Alternately, the layer closest to the wearer may be firm to provide support for the prosthesis. The front layer can include a softer silicone to produce a prosthesis with more natural drape and a better fit.

Existing breast prostheses typically feature symmetrically curved bottom which is meant to rest in the bra cup. As shown in FIGS. 2A-2B, the prosthesis is placed with in the vacant cup 16 of the user's bra 14 opposite of the natural breast cup 18. When standing, as shown in FIG. 2A, there is little upward force exerted by the user's abdomen 12 on the prosthesis 10, which results in a relatively natural appearance. However, when the user sits or bends over, as shown in FIG. 2B, the abdomen imparts an upward pressure on the prosthesis that can result in an unbalanced appearance due to the different way in which forces from the abdomen 12 are applied to the prosthesis 10 and the natural breast 18. Such forces can even cause a portion 22 of the prosthesis 10 to be pushed outside of the neckline 20 of the bra 14 (and potentially of the clothing) which can be embarrassing for the wearer.

Therefore, there is a need for a breast prosthesis that moves like a natural breast when abdominal forces are applied to it as a result of sitting or bending over.

### SUMMARY OF THE INVENTION

The disadvantages of the prior art are overcome by the present invention which, in one aspect, is a breast prosthesis for use by a user having an underarm region. The breast prosthesis includes a back surface having a footprint and a breast-shaped portion extending outwardly from the back surface so that the breast-shaped portion has a shelf. The footprint has a top neckline terminating in a medial point and a bottom shelf edge extending away from the medial point in which the bottom shelf edge of the footprint of the breast prosthesis is a substantially straight line and slopes downwardly away from the medial point when in normal use.

In another aspect, the invention is a breast prosthesis for use by a user that has an abdomen, an underarm region and a chest having a central point. The breast prosthesis has a shape so that when the user is in a position in which the user's abdomen pushes upwardly, the breast prosthesis is moved laterally outwardly from the central point on the user's chest.

In yet another aspect, the invention is a breast prosthesis for use by a user having an underarm region. The breast prosthesis includes at least one bag made of a film and a filler disposed with the bag. The breast prosthesis includes a back surface having a footprint and a breast-shaped portion extending outwardly from the back surface so that the breast-shaped portion has a shelf. The footprint has a top neckline terminating in a medial point and a bottom shelf edge extending away from the medial point in which the bottom shelf edge of the footprint of the breast prosthesis is a substantially straight line and slopes downwardly away from the medial point. A line parallel to the bottom shelf edge when the breast prosthesis is placed in a brassiere for normal use is at an acute angle relative to a horizontal axis. The breast prosthesis tapers to a distal edge having a shape that is complimentary in shape to an anatomical shape defined by the underarm region of the user. The breast prosthesis has a shallow top portion and a protruding shelf.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of a prior art breast prosthesis.

FIGS. 2A-2B are a series of schematic diagrams that demonstrate how forces are applied to a prior art prosthesis by a protruding abdomen while standing and sitting.

FIG. 3A is a front elevational view schematic diagram of a breast prosthesis adapted for use by women with protruding abdomens.

FIG. 3B is a cross-sectional view of the prosthesis shown in FIG. 3A, taken along line 3B-3B.

FIGS. 4A-4B are schematic diagrams contrasting force distributions on an existing prosthesis and a prosthesis for use by women with protruding abdomens.

FIGS. 5A-5B are a series of schematic diagrams that demonstrate how forces are applied to a prosthesis or use by women with protruding abdomens while standing and sitting.

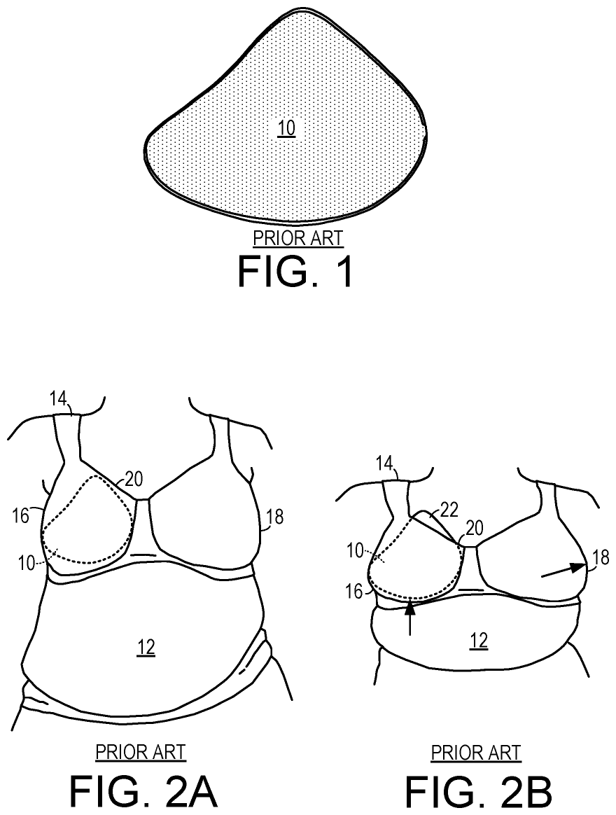

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

U.S. Pat. Nos. 4,247,351, 4,249,975 and 8,562,679, all issued to Rechenberg, describe breast prosthesis structures (including film bag/silicone rubber protheses) and methods of making breast prostheses, and are incorporated herein by reference for the purpose of disclosing prosthesis and prosthesis-making methods.

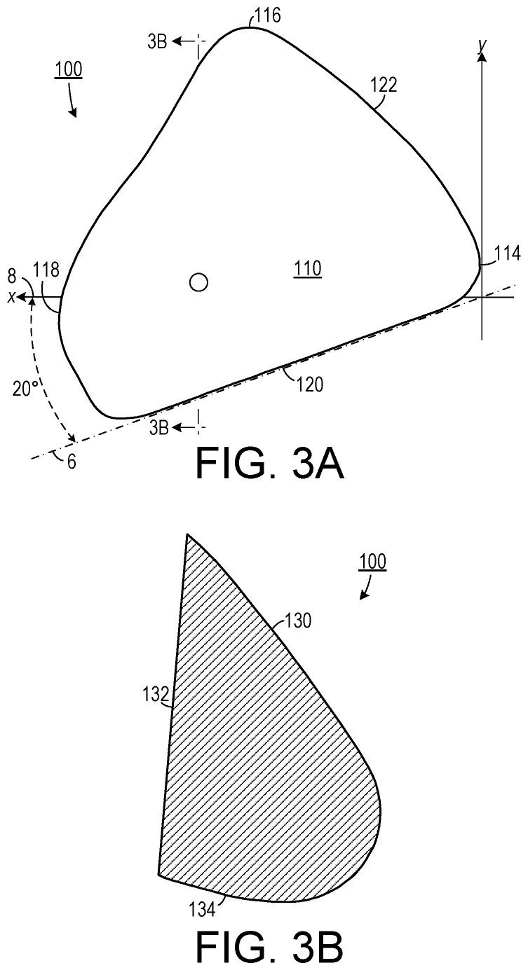

As shown in FIGS. 3A-3B, one embodiment of a breast prosthesis 100 has a footprint 110 (a shape of the breast prosthesis when projected onto a two-dimensional surface, which corresponds to an elevational view of the prosthesis and also corresponds to the shape of its back surface 132) that includes a top neckline 122 terminating in a medial point 114 and a bottom shelf edge 120 extending away from the medial point 114. (FIG. 3A shows a prosthesis 100 for use in a right-side bra cup. A mirror image of this prosthesis would be used in a left-side bra cup.) The bottom shelf edge 120 of the footprint 110 of the breast prosthesis 100 is a substantially straight line and slopes downwardly away from the medial point 114 when positioned for normal use by the user. A line 6 parallel to the bottom shelf edge 120 when the breast prosthesis is placed in a brassiere (“a bra”) for normal use is at an acute angle of about 20° relative to a horizontal axis 8. Also, the breast prosthesis 100 has a shallow top portion 130 and a protruding lower shelf 134.

The breast prosthesis 100 tapers to a distal edge 118 that has a shape that is complimentary in shape to an anatomical shape defined by the underarm region of the user. This allows the distal edge 118 to fit comfortably between the user's arm and chest. The prosthesis 100 contains an anatomical curve which allows for a snug fit to the user's chest wall. A typical asymmetric breast prosthesis embodiment extends toward the underarm. The extension toward the underarm duplicates the fullness of the unaffected breast in the case of a unilateral mastectomy. Also, in the case of a bilateral mastectomy, such an extension may be useful to complement the physique of the wearer with an abdominal protrusion. In most cases where the wearer has an abdominal protrusion, the girth of her torso lends itself to such extensions in order to appear normal and natural in her clothing.

In one embodiment, the prosthesis 100 is of the film bag/silicone rubber type. In another embodiment, it is of a synthetic rubber foam type. In this embodiment, the prosthesis 100 includes at least one bag made of a film (such as a polyurethane film) and a filler (such as silicone gel or cured silicone rubber) disposed with the bag. The breast prosthesis 100 could also be made of a unitary material, such as an open cell mechanical foam. Such an open cell mechanical foam can include a silicone matrix that defines a plurality of voids therein. (An example of a mechanical foam breast prosthesis is disclosed in U.S. Pat. No. 10,307,270, issued to Halley et al., which is incorporated herein by reference for the purpose of disclosing a mechanical foam breast prosthesis structure and methods of making the same.)

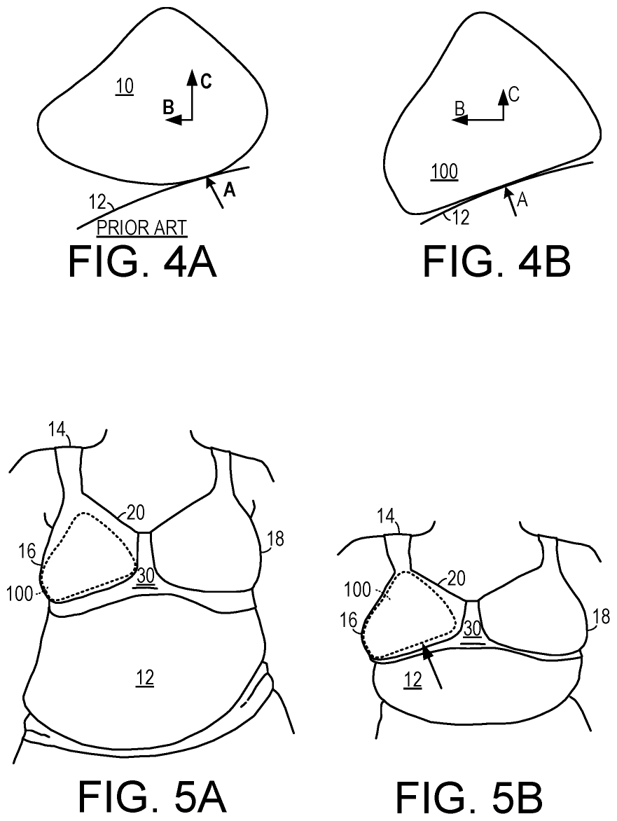

As shown in FIG. 4A, when a user's abdomen exerts force on a prior art prosthesis 10 in the direction of arrow A, the force applied to the prosthesis 10 results in an outward movement in the direction of arrow B and a substantial upward movement in the direction of arrow C. In contrast, as shown in FIG. 4B, the prosthesis 100 of the present invention increases the amount of outward movement (in the direction of arrow B) and reduces the upward movement (in the direction of arrow C), so as to more closely imitate the movement of a natural breast. (Arrows A, B and C are provided to illustrate the forces imparted on the prosthesis. They do not show actual force magnitudes and it is understood that such magnitudes in actual embodiments may differ from the vector lengths shown in FIGS. 4A and 4B.) This is demonstrated in FIG. 5A, in which the user is upright, and in FIG. 5B, in which the user is bent over or sitting. As can be seen in FIG. 5B, the shape of breast prosthesis 100 results in the breast prosthesis 100 moving laterally outwardly from the central point 30 on the user's chest when the user's abdomen 12 pushes upwardly.

This invention includes a breast prosthesis with shape designed so that when a wearer with a protruding belly sits, the form reacts as a natural breast, falling to the outside of the belly and to the side of the bra cup. The volume of the prosthesis 100 more closely matches the volume of the user's natural breast. The slope at the bottom of the prosthesis 100 may be noticeable in the bra cup, but it is not visible through clothing. The angled slope at the bottom of the breast form prosthesis 100 allows the prosthesis 100 to slide toward the side of the body, mimicking the action of the user's natural breast. It also reduces the possibility of a portion of the breast form prosthesis 100 from pushing out through the neckline of the bra.

The front of the prosthesis 100 is shallow in the profile but features a significant shelf at the bottom of the prosthesis that sits at a 20-degree angle to the horizontal axis. The font elevational view of the shelf generally does not have curve to it, but substantially follows a straight line. This shelf connects to the neckline of the prosthesis at a medial point rather than a broad curve. This point is contained within the bra cup and stays in place because of the significant shelf at the bottom of the form. The third edge of the form, adjacent to the axilla or underarm, can be a traditional curve resulting in a comfortable smooth anatomical curve to hug at the underarm. The footprint generally fits an acute scalene triangle shape in which no side corresponds to any axis. Essentially, the footprint has three sides that fit on the body at an angle so that no side is parallel to the horizontal or vertical planes.

In one embodiment, the prosthesis 100 includes at least two layers of film sealed around the periphery to create a bag (in alternative embodiments, a plurality of bags can be used) with at least one chamber having an inlet providing for the injection of materials. In making such an embodiment, the bag is placed in a mold that is complimentary in shape to the desired shape of the prosthesis. Uncured silicone gel is injected into the chamber, air is removed from the bag, the material inlet is sealed and the mold is heated in an oven so that the film is heat-formed to the shape of the mold and the uncured material is cured. The silicone gel can be formulated to be firm or soft, as required. The material can also contain additives such as pigments to affect appearance or microspheres to affect density or other physical properties.

The prosthesis 100 has the advantage of maintaining symmetry of the user appearance when she both stands and sits. It also prevents embarrassment as a result of a prosthesis protruding into view when the user sits while wearing a low neckline top.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A breast prosthesis for use by a user having an underarm region, comprising a back surface having a footprint and a breast-shaped portion extending outwardly from the back surface so that the breast-shaped portion has a shelf, the footprint having a top neckline terminating in a medial point and a bottom shelf edge extending away from the medial point in which the bottom shelf edge of the footprint of the breast prosthesis is a substantially straight line and slopes downwardly away from the medial point when positioned for normal use by the user.

2. The breast prosthesis of claim 1, wherein a line parallel to the bottom shelf edge when the breast prosthesis is placed in a brassiere for normal use is at an acute angle relative to a horizontal axis.

3. The breast prosthesis of claim 2, wherein the acute angle is about 20°.

4. The breast prosthesis of claim 1, wherein the breast prosthesis tapers to a distal edge having a shape that is complimentary in shape to an anatomical shape defined by the underarm region of the user.

5. The breast prosthesis of claim 1, wherein the breast prosthesis has a shallow top portion and a protruding lower shelf.

6. The breast prosthesis of claim 1, wherein the breast prosthesis comprises:
(a) at least one bag made of a film; and
(b) a filler disposed with the bag.

7. The breast prosthesis of claim 6, wherein the film comprises polyurethane and wherein the filler comprises silicone.

8. The breast prosthesis of claim 7, wherein the silicone comprises cured silicone.

9. The breast prosthesis of claim 1, wherein the breast prosthesis comprises a unitary material.

10. The breast prosthesis of claim 9, wherein the unitary material comprises an open cell mechanical foam.

11. The breast prosthesis of claim 10, wherein the open cell mechanical foam comprises a silicone matrix that defines a plurality of voids therein.

12. A breast prosthesis for use by a user that has an abdomen, an underarm region and a chest having a central point, wherein the breast prosthesis has a shape so that when the user is in a position in which the user's abdomen pushes upwardly, the breast prosthesis is moved laterally outwardly from the central point on the user's chest.

13. The breast prosthesis of claim 12, wherein the breast prosthesis has a footprint that includes a medial point and shelf edge, wherein the prosthesis is configured for placement in a brassiere so that the medial point is placed adjacent to the central point of the user's chest, wherein the shelf edge slopes downwardly away from the medial point, wherein a line parallel to the bottom shelf edge when the breast prosthesis is placed in a brassiere for normal use is at an acute angle relative to a horizontal axis.

14. The breast prosthesis of claim 13, wherein the acute angle is about 20°.

15. The breast prosthesis of claim 13, wherein breast prosthesis tapers to a distal edge having a shape that is complimentary in shape to an anatomical shape defined by the underarm region of the user.

16. The breast prosthesis of claim 13, wherein the breast prosthesis comprises:
(a) at least one bag made of a polyurethane film; and
(b) a silicone filler disposed with the bag, the silicone filler including cured silicone so that the breast prosthesis has a shallow top portion and a protruding shelf.

17. A breast prosthesis for use by a user having an underarm region, comprising:
(a) at least one bag made of a film; and
(b) a filler disposed with the bag,
wherein the breast prosthesis includes a back surface having a footprint and a breast-shaped portion extending outwardly from the back surface so that the breast-shaped portion has a shelf, the footprint having a top neckline terminating in a medial point and a bottom shelf edge extending away from the medial point in which the bottom shelf edge of the footprint of the breast prosthesis is a substantially straight line and slopes downwardly away from the medial point, wherein a line parallel to the bottom shelf edge when the breast prosthesis is placed in a brassiere for normal use is at an acute angle relative to a horizontal axis,
wherein the breast prosthesis tapers to a distal edge having a shape that is complimentary in shape to an anatomical shape defined by the underarm region of the user, and
wherein the breast prosthesis has a shallow top portion and a protruding shelf.

18. The breast prosthesis of claim 17, wherein the acute angle is about 20°.

19. The breast prosthesis of claim 17, wherein the film comprises polyurethane and wherein the filler comprises silicone.

20. The breast prosthesis of claim 17, wherein the breast prosthesis comprises an open cell mechanical foam that includes a silicone matrix that defines a plurality of voids therein.
